(19) United States
(12) Patent Application Publication
(10) Pub. No.: US 2009/0004101 A1
(43) Pub. Date: Jan. 1, 2009

(54) Method and Means for Enhanced Pulmonary Drug Delivery

(75) Inventors: Glyn Taylor, Cardiff (GB); Navdeep Thoofer, Cardiff (GB); Richard Evans, Cary, NC (US); Carole Evans, Cary, NC (US); Benjamin Yerxa, Raleigh, NC (US); Gregory Mossinghoff, Raleigh, NC (US)

Correspondence Address: BUCHANAN, INGERSOLL & ROONEY PC, POST OFFICE BOX 1404, ALEXANDRIA, VA, 22313-1404, US

(73) Assignee: UNIVERSITY COLLEGE CARDIFF CONSULTANTS LIMITED, Cardiff (GB)

(21) Appl. No.: 11/659,877

(22) PCT Filed: Aug. 1, 2005
(86) PCT No.: PCT/GB05/03013

§ 371 Date: Sep. 9, 2008

(30) Foreign Application Priority Data
Aug. 11, 2004 (GB) 0417886.9

(51) Int. Cl. A61K 51/00; A61K 31/7068; A61K 31/708; A61K 38/16; A61K 31/7052; A61K 35/76; A61K 38/28; C07H 19/10; C07H 19/20; A61K 38/21; A61K 38/43; A61K 38/14; A61K 38/20; A61K 49/00

(52) U.S. Cl. 424/1.11; 514/49; 514/47; 514/12; 514/44; 424/93.6; 514/3; 424/85.7; 424/85.6; 424/94.1; 514/8; 424/85.2; 424/9.1; 536/26.2

(57) ABSTRACT

The present invention provides a method of enhancing the absorption of molecules across the airway epithelium, thereby enhancing the delivery of desired therapeutic or diagnostic agents across the airway epithelium via the systemic circulation to the target site of action. The method comprises administration of a formulation comprising a pharmaceutical composition comprising a synthetic or natural nucleoside diphosphate, nucleoside triphosphate, or dinucleoside polyphosphate, together with a pharmaceutically acceptable carrier. Preferably the nucleotide is a P2Y receptor agonist which is administered at any time during treatment with a therapeutic or diagnostic agent. A preferred embodiment is a method of administering a pharmaceutical composition comprising a P2Y receptor agonist with enhanced resistance to extracellular hydrolysis, such as dinucleoside polyphosphate compounds.

### TECHNICAL FIELD

This invention relates to a method of increasing the absorptive properties of the lung by administering a nucleotide receptor agonist such as certain natural or synthetic adenine, uridine and cytidine nucleotides and dinucleotides. The compounds can be given separately or co-administered with diagnostic or therapeutic agents to enhance the absorption of molecules from the lung to the pulmonary circulation. The compounds are given by various routes of administration including inhalation, instillation and lavage, to contact the airway surface.

### BACKGROUND OF THE INVENTION

There are a number of situations where therapeutic molecules, such as proteins, peptides or other large molecules, can only effectively be administered via injection in order to achieve useful systemic levels of such a molecule. Alternatives to this invasive type of drug delivery have been investigated, including targeting the pulmonary route of delivery. Insulin, for example, has been shown by a variety of human and animal studies to be absorbed by the lungs; however, evidence in man suggests that only 20-46% of the insulin deposited in the lungs ever reaches the systemic circulation (Patton, CHEMTECH 27(12):34-38 (1997); Patton, Nature Biotechnology 16:141-143 (1998); Patton, et al, Adv. Drug Deliv. Rev. 35: 235-147 (1999)).

Although the systemic bioavailability of certain molecules from the lungs can be much greater than that from the gastrointestinal tract, methods for improving absorption following pulmonary delivery have been investigated. A number of studies have been undertaken to enhance pulmonary absorption of insulin co-administered with other agents such as surfactants (Span 85, glycocholate), protease inhibitors (nafamostat), N-lauryl-B-D-maltopyranoside, and linoleic acid-surfactant mixed micelles (Okumura, et al, 1992; Yamamoto, et al, 1993; Nelson, et al, 1996). In addition, EDTA, a compound known to increase paracellular transport, did not enhance intratracheal absorption of insulin, but it did increase the uptake of calcitonin following intratracheal administration to the lungs (Yamamoto et al, 1996).

The mechanisms of enhanced absorption across the lung are not clear, but may involve the alveolar epithelium, which is a large surface area surrounded by a bed of pulmonary capillaries. The alveoli are lined by two types of cells: Type I cells, the primary lining cells, which are flat cells with large cytoplasmic extensions; and cuboidal Type II cells (granular pneumocytes), which are thicker, contain numerous lamellar inclusion bodies and produce and secrete lung surfactant. The alveolar epithelium is believed to be the major barrier to macromolecular drug absorption into the systemic circulation (Elbert, et al., Pharmaceutical Res. 16(5):601-608 (1999)).

P2Y receptor agonists are known to induce the secretion of mucins, surfactant, and water from respiratory epithelial surfaces in the lung (Yerxa and Johnson, Drugs Future 24, 759-769 (1999); Benali, et al., Am. J. Respir. Cell. Mol. Biol. 10, 363-368 (1994); Gobran, et al., Am. J. Physiol. 267, L625-L633 (1994); Knowles, et al., New Engl. J. Med. 325, 533-538 (1991); Lethem, et al., Am. J. Respir. Cell. Mol. Biol. 9, 315-322 (1993)) In addition, P2Y receptor agonists induce tear fluid secretion and improve the lubrication and hydration of the ocular surface in dry eye disease by stimulating the release of mucins and water from the conjunctival epithelium (Hosoya, et al., J. Pharmacol. Exp. Ther. 291 (1), 53-59 (1999); Murakami, et al., Invest. Opthalmol. Vis. Sci. 41(4), S457 (ARVO Abstract 2423 (2000); Murakami, et al., Curr. Eye Res. 21(4), 782-787 (2000); Shiue, et al., Life Sci. 66(7), PL105-111 (2000); Jumblatt and Jumblatt, Exp. Eye Res. 67, 341-346 (1998))

It is now known that P2Y receptor agonists modulate all components of the mucociliary clearance system by: (1) increasing both the rate and total amount of mucin secretion by goblet cells in vitro (Lethem, et al., Am. J. Respir. Cell. Mol. Biol. 9, 315-22 (1993)); (2) increasing cilia beat frequency in human airway epithelial cells in vitro (Drutz, et al., Drug Dev. Res. 37(3), 185 (1996)); (3) increasing Cl⁻ secretion, hence, water secretion from airway epithelial cells in vitro (Mason, et al., Br. J. Pharmnacol. 103, 1649-1656 (1991); and (4) releasing surfactant from Type II alveolar cells (Gobran, Am. J. Physiol. 267, L625-L633 (1994)). Inhaled P2Y₂-receptor agonists, UTP and a novel P2Y₂ receptor agonist, INS365, can increase lung mucociliary clearance in sheep (Sabater, et al., J. Appl. Physiol. 87(6):2191-2196 (1999)). In addition to such actions, P2Y agonists have also been shown to increase intracellular Ca⁺⁺ due to stimulation of phospholipase C by the P2Y₂ receptor (Brown, et al., Mol. Pharmacol. 40, 648-655 (1991); Yerxa and Johnson, Drugs of the Future 24(7): 759-769 (1999)). U.S. Pat. Nos. 5,789,391; 5,763,447; 5,635,160; 5,935,555; 5,656,256; 5,628,984; 5,902,567; 5,292,498; 5,837,861; 5,900,407; 5,972,904; 5,981,506; 5,958,897; 5,968,913; 6,022,527; 6,133,247; and 6,143,279, and PCT International Patents WO97/29756, WO97/35591, WO96/40059, WO97/05195, WO94/08593, WO98/19685, WO98/15835, WO98/03182, WO98/03177, WO98/34942, WO98/34593, WO99/09998, WO99/32085, WO99/61012, WO 00/30629, WO 00/50024, and WO 96/40059 disclose a method of treating sinusitis, otitis media, ciliary dyskinesia, pneumonia associated with immobilization, lung disease, cystic fibrosis, dry eye disease, vaginal dryness, bronchitis, edematous retinal disorders, retinal degeneration and detachment, and gastrointestinal disease, by administrating dinucleoside polyphosphates and related compounds to a patient. These and all other U.S. patents cited and herein are specifically incorporated herein by reference in their entirety.

There exists a need for a means of promoting systemic absorption that is both safe and effective for delivery of desired molecules or therapeutic agents to various body sites. The applicants had found an unexpected result of enhanced plasma insulin levels when insulin was co-administered with a P2Y agonist. Applicants there thus motivated to further study the rule of nucleotides in systemic absorption of therapeutic molecules administered via the lung.

### SUMMARY OF THE INVENTION

The present invention provides a method of increasing the systemic absorption of molecules across the surface of the lung, said method comprising administering to a subject in need thereof a nucleotide receptor agonist in an amount effective to increase the absorption of molecules across the surface of the lung to the systemic circulation.

The present invention also provides a method of increasing the systemic absorption of molecules across the surface of the lung of a subject, said method comprising: administering to said subject a nucleotide receptor agonist in an amount effective to increase the absorption of molecules across the surface of the lung to the system circulation.

Nucleotide receptor agonists include nucleoside polyphosphates and their dinucleoside analogues. Nucleoside diphosphates useful in this application include uridine 5′-diphosphate (UDP), adenosine 5′-diphosphate (ADP), cytosine 5′-diphosphate (CDP) and their analogs of general Formula I. Nucleoside triphosphates useful in this application include uridine 5′-triphosphate (UTP), adenosine 5′-triphosphate (ATP), cytosine 5′-triphosphate (CTP) and their analogs of general Formula II; dinucleoside polyphosphates of general Formula III are also useful in this application.

### DETAILED DESCRIPTION OF THE INVENTION

The invention provides methods for enhancing pulmonary absorption using an agonist of a nucleotide receptor, which are membrane-bound proteins that specifically bind extracellular nucleotides, such as UTP and ATP. Preferably the nucleotide receptor is the P2Y purinergic receptor such as P2Y₂ receptors; such receptors activated by P2Y agonists. The present invention provides a method of facilitating drug delivery of molecules that are ineffective when given orally, or must be injected, or not optimally bioavailable even when given via inhalation. Molecules may be defined as the simplest unit of a compound that can be absorbed across the airway epithelium.

The method comprises administering to a subject in need thereof a formulation of a sterile pharmaceutical composition comprising a nucleotide receptor agonist or pharmaceutically acceptable salts thereof, together with a pharmaceutically suitable carrier. Preferably, a purinergic receptor agonist is administered in an amount effective to enhance the permeability and/or increase the absorption of molecules across the surface of the lung to the systemic circulation. An effective amount is one that significantly enhances the pulmonary absorption of molecules and may vary depending on the properties of that molecule and can be determined by various known techniques performed by those skilled in the art. An effective amount may vary depending on the properties of that molecule and can be determined by various known techniques performed by those skilled in the art.

The P2Y purinergic receptor agonist stimulates P2Y purinergic receptors, which triggers signaling pathways leading to proabsorptive effects. The nucleotide agent is administered at any time to increase the absorption of the desired molecules. Preferably the compounds are delivered as respirable particles of correct size to reach the distal lung (alveoli, small airways).

The nucleotide receptor agonist is co-administered with a therapeutic agent. The method is useful for delivering peptides, proteins, enzymes, antibodies, hormones, DNA, viruses, diagnostic agents, such as contrast, imaging, and radiolabelled compounds, and therapeutic agents, such as antimicrobial agents, antiviral agents, analgesic agents, anti-inflammatory agents, anti-neovascular agents, neuroprotectants, anti-depressants, or respiratory agents for treating any patients in need of such treatment. Therapeutic compounds suitable for such delivery are: insulin, alpha interferon, beta interferon, human growth hormone, granulocyte cell stimulating factor, epoetin alpha, epoetin beta, entanercept, aglucerase, filgrastim, lenograstim, pegaspargase, sargramostim, heparin, follicle stimulating hormone, progesterone, luprolide, estrogen, and somatrem.

The nucleotide receptor agonist is co-administered with a diagnostic agent. The method is useful for delivering contrast agents, diagnostic imaging agents and radiolabeled compounds.

A combined therapeutic approach is beneficial in reducing dose-related adverse drug effects by reducing the amount of drug required to exert a therapeutic action. In addition to enhancing safety, a combined therapeutic approach is also advantageous in increasing efficacy of treatment by enhancing the ability of a drug to reach its target site.

### DESCRIPTION OF COMPOUNDS

This invention provides a method of enhancing systemic absorption of desired molecules using a formulation comprising a pharmaceutical composition comprising nucleotide receptor agonists with a pharmaceutically acceptable carrier. Nucleotide receptor agonists include nucleoside polyphosphates and their dinucleoside analogues. Nucleoside diphosphates useful in this application include uridine 5′-diphosphate (UDP), adenosine 5′-diphosphate (ADP), cytosine 5′-diphosphate (CDP) and their analogs of general Formula I. Nucleoside triphosphates useful in this application include uridine 5′-triphosphate (UTP), adenosine 5′-triphosphate (ATP), cytosine 5′-triphosphate (CTP) and their analogs of general Formula II; dinucleoside polyphosphates of general Formula III are also useful in this application.

UDP and its analogs are depicted by general Formula Ia:

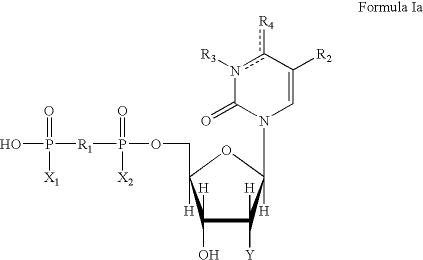

wherein:
X₁ and X₂ are each independently either O or S—;
Y is H or OH;
R₁ is O, imido, methylene, or dihalomethylene (e.g., dichloromethylene, difluoromethylene);
R₂ is H, halogen, alkyl, substituted alkyl, alkoxyl, alkenyl, or alkynyl;
R₃ is nothing, H, alkyl, acyl (including arylacyl), or arylalkyl; and
R₄ is OR′, SR′, NR′, or NR′R″, wherein R′ and R″ are independently H, alkyl, substituted alkyl, aryl, substituted aryl, arylalkyl, alkoxyl, or aryloxyl; and provided that when R₄ is double bonded from an oxygen or sulfur atom to the carbon at the 4-position of the pyrimidine ring, R′ is absent.

As used herein, the term “alkyl” refers to C₁₋₁₀ inclusive, linear, branched, or cyclic, saturated or unsaturated (i.e., alkenyl and alkynyl)hydrocarbon chains, for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, allenyl and optionally substituted arylalkenyl and arylalkyny groups. As used herein, the term “acyl” refers to an organic acid group wherein the —OH of the carboxyl group has been replaced with another substituent (i.e., as represented by RCO—, wherein R is an alkyl or an aryl group). As such, the term “acyl” specifically includes arylacyl groups. Specific examples of acyl groups include acetyl and benzoyl. As used herein, the term “aryl” refers to 5 and 6-membered hydrocarbon and heterocyclic aromatic rings. Examples of aryl groups include cyclopentadienyl, phenyl, furan, thiophene, pyrrole, pyran, pyridine, imidazole, isothiazole, isoxazole, pyrazole, pyrazine, pyrimidine, and the like. The term “alkoxyl” as used herein refers to C₁₋₁₀ inclusive, linear, branched, or cyclic, saturated or unsaturated oxo-hydrocarbon chains, including for example methoxy, ethoxy, propoxy, isopropoxy, butoxy, t-butoxy, and pentoxy. The term “aryloxyl” as used herein refers to aryloxy such as phenyloxyl, and alkyl, halo, or alkoxyl substituted aryloxyl. As used herein, the terms “substituted alkyl” and “substituted aryl” include alkyl and aryl groups, as defined herein, in which one or more atoms or functional groups of the aryl or alkyl group are replaced with another atom or functional group, for example, halogen, aryl, alkyl, alkoxy, hydroxy, nitro, amino, alkylamino, dialkylamino, sulfate, and mercapto. The terms “halo,” “halide,” or “halogen” as used herein refer to fluoro, chloro, bromo, and iodo groups.

Formula Ia compounds, for example, include: uridine 5′-diphosphate (UDP); uridine 5′-O-(2-thiodiphosphate) (UDPβS); 5-bromouridine 5′-diphosphate (5-BrUDP); 5-(1-phenylethynyl)-uridine 5′-diphosphate (5-(1-phenylethynyl)UDP); 5-methyluridine 5′-diphosphate (5-methylUDP); 4-hexylthiouridine 5′-diphosphate (4-hexylthioUDP); 4-mercaptouridine 5′-diphosphate (4-mercaptoUDP); 4-methoxyuridine 5′-diphosphate (4-methoxyUDP); 4-(N-morpholino)uridine 5′-diphosphate (4-(N-morpholino)UDP; 4-hexyloxyuridine 5′-diphosphate (4-hexyloxyUDP); N,N-dimethylcytidine 5′-diphosphate (N,N-dimethylCDP); N-hexylcytidine 5′-diphosphate (N-hexylCDP); and N-cyclopentylcytidine 5′-diphosphate (N-cyclopentylCDP).

Preferred compounds of Formula Ia include UDP and UDPβS and 4-thio UDP. Certain compounds of Formula Ia (e.g., UDP, dUDP, UDPβS, and 4-mercaptoUDP) are known and may be made in accordance with known procedures or variations thereof, which will be apparent to those skilled in the art. For example, the identification and preparation of certain thiophosphate analogues of nucleoside diphosphates (such as UTP-β-S) are set forth in U.S. Pat. No. 3,846,402 and Goody and Eckstein (J. Am. Chem. Soc. 93: 6252-6257 (1971)). Alternatively, UDP, and other analogs thereof are also commercially available from vendors such as Sigma (St. Louis, Mo.) and Pharmacia (Uppsala, Sweden).

ADP and its analogs are depicted by general Formula Ib:

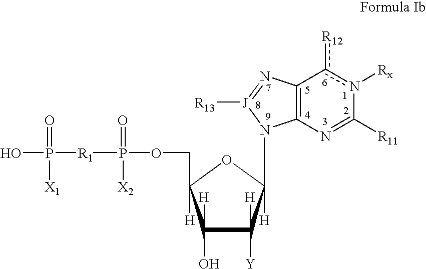

wherein:
R₁, X₁, X₂ and Y are defined as in Formula Ia;

wherein:
R₁₁ is hydrogen, chlorine, amino, monosubstituted amino, disubstituted amino, alkylthio, arylthio, or aralkylthio, wherein the substituent on sulfur contains up to a maximum of 20 carbon atoms, with or without unsaturation;
R₁₂ is hydroxy, alkenyl, oxo, amino, mercapto, thione, alkylthio, arylthio, aralkylthio, acylthio, alkyloxy, aryloxy, aralkyloxy, acyloxy, monosubstituted alkylamino, heterocyclic, monosubstituted cycloalkylamino, monosubstituted aralkylamino, monosubstituted arylamino, diaralkylamino, diarylamino, dialkylamino, acylamino, or diacylamino;
R_{X} is O, H, or is absent;
R₁₂ and R_{X} are optionally taken together to form a 5-membered fused imidazole ring of 1, N⁶-ethenoadenine derivatives, optionally substituted on the 4- or 5-positions of the etheno moiety with alkyl, aryl, nitroaryl, haloaryl, aralkyl, or alkoxy moieties as defined below;
R₁₃ is hydrogen, azido, alkoxy, aryloxy, aralkyloxy, alkylthio, arylthio, or aralkylthio as defined below; or T(C₁₋₆alkyl)OCONH(C₁₋₆alkyl)W— wherein T and W are independently amino, mercapto, hydroxy, or carboxyl; or pharmaceutically acceptable esters, amides or salts thereof;
J is carbon or nitrogen, with the provision that when J is nitrogen, R₁₃ is not present;

wherein the alkyls are straight-chain, branched or cyclic;

wherein the aryl groups are optionally substituted with lower alkyl, aryl, amino, mono- or dialkylamino, NO₂, N₃, cyano, carboxylic, amido, sulfonamido, sulphonic acid, phosphate, or halo groups;

Particularly preferred compounds of Formula Ib include 5′-adenosine diphosphate (ADP) and 2-methyl-SADP.

CDP and its analogs are depicted by general Formula Ic:

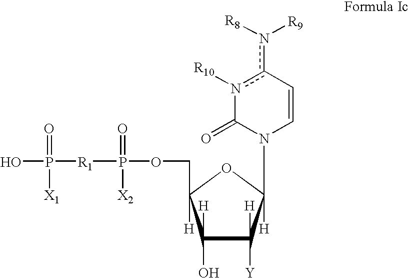

wherein:
R₁, X₁, X₂ and Y are defined as in Formula Ia;
R₈ and R₉ are H while R₁₀ is nothing and there is a double bond between N-3 and C-4 (cytosine), or
R₈, R₉ and R₁₀ taken together are —CH═CH—, forming a ring from N-3 to N-4 with a double bond between N-4 and C-4 (3,N⁴-ethenocytosine); optionally, the hydrogen of the 4- or 5-position of the etheno ring is substituted with alkyl, substituted alkyl, aryl, substituted aryl (heteroaryl, nitroaryl, etc.), alkoxyl, nitro, halogen, or azido.

UTP and its analogs are depicted by general Formula IIa;

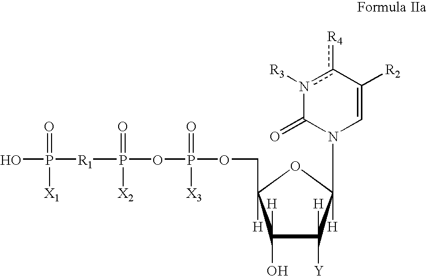

wherein:
X₁, X₂ and X₃ are each independently either O⁻ or S⁻,
Y is H or OH;
R₁, R₂, R₃ and R₄ are defined as in Formula Ia.

Preferably, X₂ and X₃ are O⁻, R₁ is oxygen or imido, and R₂ is H. Particularly preferred compounds of Formula Ia include uridine 5′-triphosphate (UTP) and uridine 5′-O-(3-thiotriphosphate) (UTPγS).

ATP and its analogs are depicted by general Formula IIb:

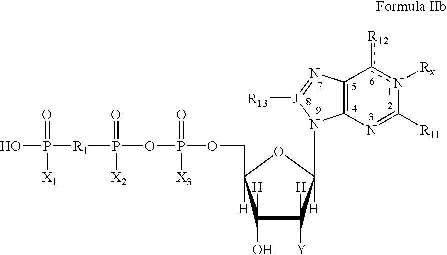

wherein:
R₁, X₁, X₂, X₃ and Y are defined as in Formula Ia;

wherein:
R₁₁ is hydrogen, chlorine, amino, monosubstituted amino, disubstituted amino, alkylthio, arylthio, or aralkylthio, wherein the substituent on sulfur contains up to a maximum of 20 carbon atoms, with or without unsaturation;
R₁₂ is hydroxy, alkenyl, oxo, amino, mercapto, thione, alkylthio, arylthio, aralkylthio, acylthio, alkyloxy, aryloxy, aralkyloxy, acyloxy, monosubstituted alkylamino, heterocyclic, monosubstituted cycloalkylamino, monosubstituted aralkylamino, monosubstituted arylamino, diaralkylamino, diarylamino, dialkylamino, acylamino, or diacylamino;
R_{X} is O, H, or is absent;
R₁₂ and R_{X} are optionally taken together to form a 5-membered fused imidazole ring of 1, N⁶-ethenoadenine derivatives, optionally substituted on the 4- or 5-positions of the etheno moiety with alkyl, aryl or aralkyl moieties as defined below;
R₁₃ is hydrogen, azido, alkoxy, aryloxy, aralkyloxy, alkylthio, arylthio, or aralkylthio as defined below; or T(C₁₋₆alkyl)OCONH(C₁₋₆alkyl)W— wherein T and W are independently amino, mercapto, hydroxy, or carboxyl; or pharmaceutically acceptable esters, amides or salts thereof;
J is carbon or nitrogen, with the provision that when J is nitrogen, R₁₃ is not present;

wherein the alkyls are straight-chain, branched or cyclic; and

wherein the aryl groups are optionally substituted with lower alkyl, aryl, amino, mono- or dialkylamino, NO₂, N₃, cyano, carboxylic, amido, sulfonaido, sulphonic acid, phosphate, or halo groups.

CTP and its analogs are depicted by general Formula IIc:

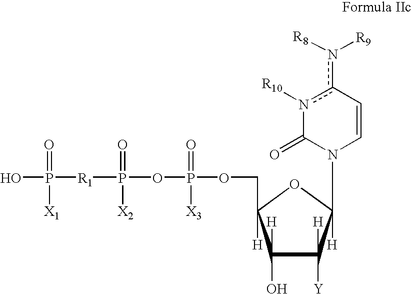

wherein:
R₁, X₁, X₂, X₃ and Y are defined as in Formula IIa, and
R₈, R₉ and R₁₀ are defined as in Formula Ic.

Preferred compounds of Formula IIc include cytidine 5′-triphosphate (CTP) and 4-nitrophenyl ethenocytidine 5′-triphosphate.

For simplicity, Formulae I and II, herein illustrate the active compounds in the naturally occurring D-configuration, but the present invention also encompasses compounds in the L-configuration, and mixtures of compounds in the D- and L-configurations, unless otherwise specified. The naturally occurring D-configuration is preferred.

Another embodiment of the invention is directed to compounds of general Formula III or the pharmaceutically acceptable non-toxic salts thereof:

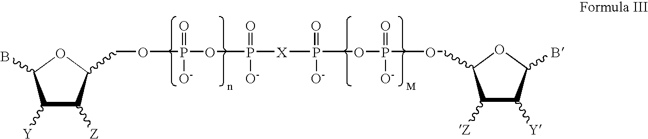

wherein:
X is oxygen, methylene, dihalomethylene (with difluoromethylene and dichloromethylene preferred), or imido;
n=0, 1 or 2;
m=0, 1 or 2;
n+m=0, 1, 2, 3 or 4;
Z=H or OH;
Z′=H or OH;
Y=H or OH;
Y′=H or OH; and
B and B′ are each independently a purine residue or a pyrimidine residue, as defined in Formula IIIa and IIIb, respectively, linked through the 9- or 1-position, respectively.

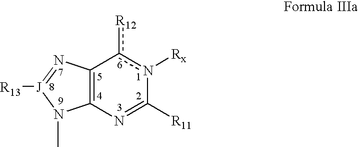

wherein:

R₁₁ is hydrogen, chlorine, amino, monosubstituted amino, disubstituted amino, alkylthio, arylthio, or aralkylthio, wherein the substituent on sulfur contains up to a maximum of 20 carbon atoms, with or without unsaturation;

R₁₂ is hydroxy, alkenyl, oxo, amino, mercapto, thione, alkylthio, arylthio, aralkylthio, acylthio, alkyloxy, aryloxy, aralkyloxy, acyloxy, monosubstituted alkylamino, heterocyclic, monosubstituted cycloalkylamino, monosubstituted aralkylamino, monosubstituted arylamino, diaralkylamino, diarylamino, dialkylamino, acylamino, or diacylamino;

R_{X} is O, H, or is absent;

R₁₂ and R_{X} are optionally taken together to form a 5-membered fused imidazole ring of 1, N⁶-ethenoadenine derivatives, optionally substituted on the 4- or 5-positions of the etheno moiety with alkyl, aryl or aralkyl moieties as defined below;

R₁₃ is hydrogen, azido, alkoxy, aryloxy, aralkyloxy, alkylthio, arylthio, or aralkylthio as defined below; or T(C₁₋₆alkyl)OCONH(C₁₋₆alkyl)W— wherein T and W are independently amino, mercapto, hydroxy, or carboxyl; or pharmaceutically acceptable esters, amides or salts thereof;

J is carbon or nitrogen, with the provision that when J is nitrogen, R₁₃ is not present;

wherein the alkyls are straight-chain, branched or cyclic;

wherein the aryl groups are optionally substituted with lower alkyl, aryl, amino, mono- or dialkylamino, NO₂, N₃, cyano, carboxylic, amido, sulfonamido, sulphonic acid, phosphate, or halo groups;

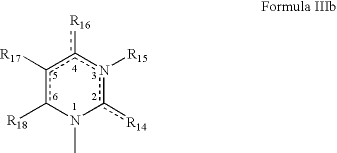

wherein:
R₁₄ is hydroxy, oxo, mercapto, thione, amino, cyano, C₇₋₁₂arylalkoxy, C₁₋₆ alkylthio, C₁₋₆ alkoxy, C₁₋₆ alkylamino, or diC₁₋₄alkylamino, wherein the alkyl groups are optionally linked to form a heterocycle;
R₁₅ is hydrogen, acetyl, benzoyl, C₁₋₆ alkyl, C₁₋₅ alkanoyl, aroyl, or absent;
R₁₆ is hydroxy, oxo, mercapto, thione, C₁₋₄alkoxy, C₇₋₁₂arylalkoxy, C₁₋₆alkylthio, S-phenyl, arylthio, aralkylthio triazolyl, amino, C₁₋₆alkylamino, C₁₋₅ disubstituted amino, or di-C₁₋₄alkylamino, wherein said dialkyl groups are optionally linked to form a heterocycle or linked to form a substituted ring, such as morpholino, pyrrolo, etc.; or
R₁₅ and R₁₆ taken together form a 5-membered fused imidazole ring between positions 3 and 4 of the pyrimidine ring and form a 3,N⁴-ethenocytosine derivative, wherein said etheno moiety is optionally substituted on the 4- or 5-positions with C₁₋₄ alkyl, phenyl or phenyloxy; wherein at least one hydrogen of said C₁₋₄ alkyl, phenyl or phenyloxy is optionally substituted with halogen, hydroxy, C₁₋₄ alkoxy, C₁₋₄ alkyl, C₆₋₁₀ aryl, C₇₋₁₂ arylalkyl, carboxy, cyano, nitro, sulfonamido, sulfonate, phosphate, sulfonic acid, amino, C₁₋₄ alkylamino, and di-C₁₋₄ alkylamino, wherein said dialkyl groups are optionally linked to form a heterocycle;
R₁₇ is hydrogen, hydroxy, cyano, nitro, C₁₋₆ alkyl, phenyl, substituted C₂₋₈ alkynyl, halogen, substituted C₁₋₄ alkyl, CF₃, C₂₋₃ alkenyl, C₂₋₃ alkynyl, allylamino, bromovinyl, ethyl propenoate, propenoic acid, or C₂₋₈ alkenyl; or
R₁₆ and R₁₇ together form a 5 or 6-membered saturated or unsaturated ring bonded through N or O or S at R₆; such ring optionally contains substituents that themselves contain functionalities; and
R₁₈ is hydrogen, amino, di-C₁₋₄alkylamino, C₁₋₄alkoxy, C₇₋₁₂arylalkoxy, C₁₋₄alkylthio, C₇₋₁₂arylalkylthio, carboxamidomethyl, carboxymethyl, methoxy, methylthio, phenoxy, or phenylthio; provided that when R₁₈ is amino or substituted amino, R₇ is hydrogen.

The furanosyl moieties are as depicted in the D-configuration, but may be L-, or D- and L-. The D-configuration is preferred. The nucleoside residue can be an alpha- or beta- and D- or L-configurations, but most preferably the beta-D-configuration. The furanosyl moieties include ribofuranosyl, 2′-deoxyribofuranosyl, 3′-deoxyribofuranosyl, 2′,3′-dideoxyribofuranosyl, arabinofuranosyl, 3′-deoxyarabinofuranosyl, xylofuranosyl, 2′-deoxyxylofuranosyl, and Iyxofuranosyl.

In the general structure of Formulae IIIa, the dotted lines are intended to indicate the presence of single or double bonds in these positions; the relative positions of the double or single bonds being determined by whether the R₁₂ and R_{X} substituents are capable of keto-enol tautomerism.

In the general structure of Formulae IIIb, the dotted lines in the 2- to 6-positions are intended to indicate the presence of single or double bonds in these positions; the relative positions of the double or single bonds being determined by whether the R₁₄, R₁₅, R₁₆, R₁₇, and R₁₈ substituents are capable of keto-enol tautornerism.

In the general structures of Formulae Ia, Ib, Ic, IIa, IIb, IIc, III, IIIa, and IIIb above, the acyl groups comprise alkanoyl or aroyl groups. The alkyl groups contain 1 to 8 carbon atoms, particularly 1 to 4 carbon atoms optionally substituted by one or more appropriate substituents, as described below. The aryl groups including the aryl moieties of such groups as aryloxy are preferably phenyl groups optionally substituted by one or more appropriate substituents, as described below. The above-mentioned alkenyl and alkynyl groups contain 2 to 8 carbon atoms, particularly 2 to 6 carbon atoms, e.g., ethenyl or ethynyl, optionally substituted by one or more appropriate substituents as described below.

Appropriate substituents on the above-mentioned alkyl, alkenyl, alkynyl, and aryl groups are selected from halogen, hydroxy, C₁₋₄ alkoxy, C₁₋₄ alkyl, C₆₋₁₂ aryl, C₆₋₁₂ arylalkoxy, carboxy, cyano, nitro, sulfonamido, sulfonate, phosphate, sulfonic, amino and substituted amino wherein the amino is singly or doubly substituted by a C₁₋₄ alkyl, and when doubly substituted, the alkyl groups optionally being linked to form a heterocycle.

Substituted derivatives of adenine include adenine 1-oxide; 1,N⁶-(4- or 5-substituted etheno) adenine; N⁶-substituted adenine; or N-substituted 8-aminoadenine, wherein said substituted groups are chosen from among: arylalkyl (C₁₋₆) groups with the aryl moiety optionally functionalized as described below; alkyl; and alkyl groups with functional groups therein, such as: ([6-aminohexyl]carbamoylmethyl)-, ω-acylated-amino(hydroxy, thiol and carboxy)alkyl(C₂₋₁₀)— and their ω-acylated-amino (hydroxy, thiol and carboxy) derivatives wherein the acyl group is chosen from among, but not limited to, acetyl, trifluoroacetyl, benzoyl, substituted-benzoyl, etc., or the carboxylic moiety is present as its ester or amide derivative, for example, the ethyl or methyl ester or its methyl, ethyl or benzamido derivative. The ω-amino(hydroxy, thiol) moiety may be alkylated with a C₁₋₄ alkyl group.

A preferred nucleotide agonist is a hydrolysis-resistant agonist. A hydrolysis-resistant agonist is a nucleotide with a modified phosphate ester backbone, e.g. a methylene, imido or other group that protects the phosphate ester bonds from being readily hydrolyzed. Dinucleotides are also resistant to hydrolysis due to a lack of a terminal phosphate group. Certain dinucleotides are especially resistant to hydrolysis. For example, P¹-(cytosine 5′)-P⁴-(uridine 5′)tetraphosphate is more resistant in comparison with P¹,P⁴-di(uridine 5′-)tetraphosphate. Furthermore, groups placed on the end of the phosphate chain imparts some stability against hydrolysis, e.g. simple alkyl phosphate esters (methyl, ethyl, benzyl, etc.) or a thio group (e.g. UTPgammaS).

Dinucleoside polyphosphates of general Formula III include dinucleoside tetraphosphates selected from the group consisting of P¹P⁴-di(uridine 5′-)tetraphosphate; P¹-(cytosine 5′)-P⁴-(uridine 5′)tetraphosphate; P¹,P⁴-di(adenosine 5′-)tetraphosphate; P¹ (adenosine 5′)-P⁴-(uridine 5′-)tetraphosphate; P¹-(adenosine 5′)-P⁴-(cytosine 5′-)tetraphosphate; P¹,P⁴-di(ethenoadenosine)tetraphosphate; P¹-(uridine 5′-)-P⁴-(thymidine 5′-) tetraphosphate; P¹-(adenosine 5′)-P⁴-(inosine 5′-)tetraphosphate; P¹,P⁴-di(uridine 5′-)P²,P³-methylenetetraphosphate; P¹,P⁴-di(uridine 5, P²,P³-difluoromethylenetetraphosphate); P¹,P⁴-di(uridine 5′-P²,P³-imidotetraphosphate); P¹,P⁴-di(4-thiouridine 5′-tetraphosphate); P¹,P⁴-di(3,N⁴-ethenocytidine 5′-) tetraphosphate; P¹, P⁴-di(imidazo[1,2-c]pyrimidine-5(6H)-one-2-(3-nitro)-phenyl-6-β-D-ribofuranoside 5′-)tetraphosphate, tetraammonium salt; P¹-(inosine 5′-)P⁴-(uridine 5′-)tetraphosphate; P¹-(4-thiouridine 5′-)P⁴-(uridine 5′-)tetraphosphate; P¹-(cytosine β-D-arabinofuranoside 5′-)P⁴-(uridine 5′-) tetraphosphate; P¹-(uridine 5′-) P⁴-(xanthosine 5′-)tetraphosphate; P¹-(2′-deoxyuridine 5′-)-P⁴-(uridine 5′-) tetraphosphate; P¹-(3′-azido-3′-deoxythymidine 5′-)-P⁴-(uridine 5′-)tetraphosphate; P¹,P⁴-di(3′-azido-3′-deoxythymidine 5′-)tetraphosphate₂P₄; P¹,P⁴-di(3′-azido-3′-deoxythymidine 5′-)tetraphosphate; 2′ (3′)-benzoyl-P¹,P⁴-di(uridine 5′-)tetraphosphate; P¹,P⁴-di(2′,3′)-benzoyl uridine 5′-) tetraphosphate; P¹-(2′-deoxyguanosine 5′-)P⁴-(uridine 5′-)tetraphosphate; P¹-(2′-deoxyadenosine 5′-)P⁴-(uridine 5′-)tetraphosphate; P¹-(2′-deoxyinosine 5′-)P⁴-(uridine 5′-)tetraphosphate; P¹-(2′-deoxycytidine 5′-)P⁴-(uridine 5′-)tetraphosphate; P¹-(4-thiouridine 5′-)P⁴-(uridine 5′-)tetraphosphate; P¹-(8-azaadenosine-5′-)P⁴-(uridine 5′-) tetraphosphate; P¹-(6-mercaptopurine riboside 5′-)P⁴-(uridine 5′-)tetraphosphate; P¹-(6-mercaptopurine riboside 5′-)P⁴-(2′-deoxyuridine 5′-)tetraphosphate; P¹-(4-thiouridine 5′-)P⁴-(arabinocytidine 5′-)tetraphosphate; P¹-(adenosine 5′-)P⁴-(4-thiomethyluridine 5′-) tetraphosphate; P¹-(2′-deoxyadenosine 5′-)P⁴-(6-thiohexylpurine riboside 5′-) tetraphosphate, and P¹-(6-eicosanyloxypurine riboside 5′-)P⁴-(uridine 5′-) tetraphosphate.

In addition, dinucleoside polyphosphates of general Formula III include dinucleoside triphosphates selected from a group consisting of: P¹P³-di(uridine 5′-)triphosphate; P¹-(cytosine 5′)-P³-(uridine 5′-)triphosphate; P¹,P³-di(adenosine 5′-)triphosphate; P¹-(adenosine 5′)-P³-(uridine 5′-)triphosphate; P¹-(adenosine 5′)-P³-(cytosine 5′-)triphosphate; P¹,P³-di(ethenoadenosine)triphosphate; P¹-(uridine 5′)-P³-(thymidine 5′-)triphosphate; P¹-(adenosine 5′)-P³-(inosine 5′-)triphosphate; P¹,P³-di(uridine 5,-)P²,P³-methylenetriphosphate; P¹,P³-di(uridine 5′-P²P³-difluoromethylenetriphosphate); P¹,P³-di(uridine 5′-P²,P³-imidotriphosphate); P¹,P³-di(4-thiouridine 5′-triphosphate); P¹,P³-di(3,N⁴-ethenocytidine 5′-)triphosphate; P¹,P³-di(imidazo[1,2-c]pyrimidine-5(6H)-one-2-(3-nitro)-phenyl-6-β-D-ribofuranoside 5′-)triphosphate, tetraammonium salt; P¹-(inosine 5′-)P³-(uridine 5′-)triphosphate; P¹-(4-thiouridine 5′-)P³-(uridine 5′-) triphosphate; P¹-(cytosine β-D-arabinofuranoside 5′-)P³-(uridine 5′) triphosphate; P¹-(uridine 5′-)P³-(xanthosine 5′-)triphosphate; P¹-(2′-deoxyuridine 5′-)-P³-(uridine 5′-)triphosphate; P¹-(3′-azido-3′-deoxythymidine 5′-)-P³-(uridine 5′-) triphosphate; P¹,P³-di(3′-azido-3′-deoxythymidine 5′-)triphosphate; P¹,P³-di(3′-azido-3′-deoxythymidine 5′-)triphosphate; 2′(3′)-benzoyl-P¹,P³-di(uridine 5′-)triphosphate; P¹,P³-Di(2′(3′)-benzoyl uridine 5′-) triphosphate; P¹-(2′-deoxyguanosine 5′-)P³-(uridine 5′-)triphosphate; P¹-(2′-deoxyadenosine 5′-)P³-(uridine 5′-)triphosphate; P¹-(2′-deoxyinosine 5′-)P³-(uridine 5′-)triphosphate; P¹-(2′-deoxycytidine 5′-)P³-(uridine 5′-)triphosphate; P¹-(4-thiouridine 5′-)P³-(uridine 5′-)triphosphate; P¹-(8-azaadenosine-5′-)P³-(uridine 5′-) triphosphate; P¹-(6-mercaptopurine riboside 5′-)P³-(uridine 5′-)triphosphate; P¹-(6-mercaptopurine riboside 5′-)P³-(2′-deoxyuridine 5′-)triphosphate; P¹-(4-thiouridine 5′-)P³-(arabinocytidine 5′-)triphosphate; P¹-(adenosine 5′-)P³-(4-thiomethyluridine 5′-) triphosphate; P¹-(2′-deoxyadenosine 5′-)P³-(6-thiohexylpurine riboside 5′-) tetraphosphate; and P¹-(6-eicosanyloxypurine riboside 5′-)P³-(uridine 5′-) triphosphate.

Furthermore, dinucleoside polyphosphates of general Formula II include compounds selected from a group consisting of: P¹-(uridine 5′-)P²-(4-thiouridine 5′-) diphosphate; P¹,P⁵-di(uridine 5′-)pentaphosphate; and P¹,P⁶-di(uridine 5′-) hexaphosphate.

Compounds encompassed by the preferred embodiment of the present invention can be prepared by condensation of a nucleoside mono-, di-, or triphosphate, activated with a condensing agent such as, but not limited to, carbonyldimidazole or dicyclohexylcarbodiimide, with a second molecule of the same or a different mono-, di-, or triphosphate to form the desired dinucleotide polyphosphate. Another method of preparation is the sequential condensation of a nucleoside phosphate, activated as above, with a non-nucleoside mono-, di- or polyphosphate moiety, such as, but not limited, to a monophosphate or pyrophosphate anion to yield the desired dinucleotide polyphosphate, the non-isolated intermediate in such a case being a mononucleotide polyphosphate. Yet another preparative approach is the sequential condensation of a mono-, di- or polyphosphate moiety, activated as mentioned above, or in the form of an acid halide or other derivative reactive toward nucleophilic displacement, with a nucleoside phosphate or polyphosphate to yield the desired dinucleotide polyphosphate. The desired dinucleotide polyphosphate may be formed by modification of a pre-formed dinucleotide polyphosphate by substitution or derivatization of a moiety or moieties on the purine, pyrimidine or carbohydrate ring. Nucleoside phosphates used as starting materials may be commercially available, or may be made from the corresponding nucleosides by methods well known to those skilled in the art. Likewise, where nucleosides are not commercially available, they may be made by modification of other readily available nucleosides, or by synthesis from heterocyclic and carbohydrate precursors by methods well known to those skilled in the art.

Those having skill in the art will recognize that the starting materials may be varied and additional steps employed to produce compounds encompassed by this embodiment of the present invention, as demonstrated by the following examples. In some cases protection of certain reactive functionalities may be necessary to achieve some of the above transformations. In general, the need for such protecting groups will be apparent to those skilled in the art of organic synthesis as well as the conditions necessary to attach and remove such groups.

The compounds of the present invention also encompass their non-toxic pharmaceutically acceptable salts, such as, but not limited to, an alkali metal salt such as sodium or potassium; an alkaline earth metal salt such as manganese, magnesium or calcium; or an ammonium or tetraalkyl ammonium salt, i.e., NX₄⁺ (wherein X is C₁₋₄). Pharmaceutically acceptable salts are salts that retain the desired biological activity of the parent compound and do not impart undesired toxicological effects. The present invention also encompasses the acylated prodrugs of the compounds disclosed herein. Those skilled in the art will recognize various synthetic methodologies, which may be employed to prepare non-toxic pharmaceutically acceptable salts and acylated prodrugs of the compounds (International Patent Nos. WO 96/40059, WO 96/02554A1, WO-A-9815563, and WO 98/55494; Theoclitou, et al., J. Chem. Soc. Perkin Trans. 1, 2009-2019 (1996); Guranowski, et al., Nucleosides and Nucleotides 14, 731-734 (1995); Visscher, et al., Nucleic Acids Research 20, 5749-5752 (1992); Holler, et al., Biochemistry 22, 4924-4933 (1983); Orr, et al., Biochem. Pharmacol. 673-677 (1988); Plateau, et al., Biochemistry 24, 914-922 (1985); Hagmeier, et al., J. Chromatography 237, 174-177 (1982); Scheffzek, et al., Biochemistry 35, 9716-9727 (1996); Stridh, et al., Antiviral Res., 97-105 (1981); Tarasova, et al., Chem. Abs. 110, 154770 (1988); Hata, et al., Chem. Lett., 987-990 (1976); Huhn, et al., 28, 1959-1970 (1993); Tumanov, et al., Chem. Abs. 109-6867d (1987); Pintor, et al., Molecular Pharmacology 51, 277-284 (1997); and U.S. Pat. Nos. 4,855,304; 5,635,160; 5,495,550; and 5,681,823).

The pharmaceutical utility of compounds of this invention is indicated by the inositol phosphate assay for P2Y₂ and other P2Y receptor activity. This widely used assay, as described in Lazarowski, et al. (1995) (Brit. J. Pharm. 116, 1619-27), relies on the measurement of inositol phosphate formation as a measurement of activity of compounds activating receptors linked via G-proteins to phospholipase C. The efficacy of these compounds is reflected in their ability to increase the absorptive properties of the lungs.

Dosage levels of the order of from about 10⁻⁷ M to about 10⁻¹ M, preferably in the range 10⁻⁵ to 10⁻¹ M, are useful in enhancing systemic absorption of molecules from the lung. The effective dose ranges between about 0.01 to about 1000 mg, preferably between about 0.1 to about 100 mg, and most preferably between about 0.5 to about 50 mg for single doses. The amount of active ingredients that may be combined with the carrier materials to produce a single dosage form will vary depending upon the host treated and the particular mode of administration. It will be understood, however, that the specific dose level for any particular patient will depend upon a variety of factors, including the activity of the specific compound employed, the age, body weight, general health, sex, diet, time of administration, route of administration, and rate of excretion, drug combination and the severity of the particular disease undergoing therapy, and can be determined by those skilled in the art.

Though the compounds of the present invention are primarily concerned with the treatment of human subjects, they may also be employed for the treatment of other mammalian subjects such as dogs and cats for veterinary purposes.

### Administration of Novel Compounds

There are various methods of administering the therapeutic compound and the enhancer compound to the lungs. The compounds are administered systemically in a form selected from the group consisting of: an aerosol suspension of respirable particles; a liquid or liquid suspension for administration as nose drops or nasal spray; a nebulized liquid for administration to oral or nasopharyngeal airways; an oral form; an injectable form; a suppository form; and a transdermal patch or a transdermal pad; such that a therapeutically effective amount of said compound contacts the airway epithelium of said subject via systemic absorption and circulation

One such means involve an aerosol mixture of respirable particles comprised of the active compounds, which the subject inhales. The therapeutic compound is absorbed into the bloodstream via the lungs in a pharmaceutically effective amount. The respirable particles may be liquid or solid, with a particle size sufficiently small to pass through the mouth and larynx upon inhalation; in general, particles ranging from about 1 to 10 microns, but more preferably 1-5 microns, in size are considered respirable.

Another means of delivering the therapeutic compound and the enhancer compound to the lungs of the subject involve administering a liquid/liquid suspension in the form of nasal drops of a liquid formulation, or a nasal spray of respirable particles which the subject inhales. Liquid pharmaceutical compositions of the active compound for producing a nasal spray or nasal drops are prepared by combining the active compounds with a suitable vehicle, such as sterile pyrogen free water or sterile saline by techniques known to those skilled in the art.

Another means of administering the active compound would involve direct intra-operative instillation of a gel, cream, or liquid suspension form of a therapeutically effective amount of the active compounds. Such intra-operative instillation could take place during bronchoscopy, thoracotomy or during surgery to remove non-functioning, hyper-inflated sections of the lung, as is sometimes required in advanced stages of bronchitis, bronchiectasis or emphysema.

Yet another method of administering the active compound is by bronchiolar lavage, which is used as a research and a clinical tool and is a safe and informative diagnostic tool.

The invention is illustrated further by the following examples of treatment which are not to be construed as limiting the scope of the specific procedures describing them.

### EXAMPLES

### Example 1

### Enhanced Pulmonary Delivery of Insulin Co-Administered with P2Y Receptor Agonists to Rabbit Lungs In Vivo

The effects of the P2Y₂ receptor agonists, UTP and Up₄U, on absorption of human insulin from the lung were investigated following intratracheal administration via an endotracheal tube to anesthetized rabbits according to methods generally described by [insert best general ref here]. Briefly, New Zealand white rabbits were anesthetized with Hypnovel (Roche, Welwyn Garden City, UK), 0.3 mg/kg i.v. via cannula in the ear, and Hypnorrn (Janssen Animal Health, Grove, Oxford, UK), 0.1 mg/kg via intramuscular injection. Once intubated with a polyethylene endotracheal tube (i.d. 4 mm, o.d. 5 mm; Portex, UK) coated with a layer of xylocalne gel (Astra Pharmaceuticals, Kings Langley, UK), a second, smaller polyethylene dosing tube was inserted inside the endotracheal tube to the point of the bifurcation of the trachea. Then human insulin 10 U/kg (prepared with 99 mTc-labelled tin colloid suspension and 0.9% saline) either alone or in combination with P2Y receptor agonist solutions (see tables for concentrations) in a maximum volume of 0.5 mL was administered through the dosing tube via syringe. Both tubes were immediately removed and venous blood samples (ca. 0.35 mL) were taken at 6, 13, 20, 30, 40, 50, 60, 70, 80, 90, 120, 150, 180, 240, 300 and 360 min post dose and analyzed for insulin concentration using a radioimmunoassay kit (ICN Pharmaceuticals, Bryan, Ohio, USA).

The table below shows the results (mean±SD; n=4) of selected pharmacokinetic parameters after dosing with insulin alone, or with 0.34 or 1.5 mg UTP.

| | | | |
|---|---|---|---|
| Insulin dose (U/kg) | 10 | 10 | 10 |
| UTP dose | — | 0.34 | 1.5 |
| (mg) | | | |
| UTP concentration | — | 3.4 mg/mL | 15 mg/mL |
| (mg/mL) | | | |
| t_{1/2} (min) | 44.9 ± 11.51 | 56.1 ± 17.80 | 61.1 ± 10.87 |
| Cₘₐₓ (μU/mL) | 440 ± 44.6 | 828 ± 42.4 | 1515 ± 297 |
| F (%) | 4.48 ± 0.13 | 11.8 ± 2.08 | 16.6 ± 4.90 |

The table below shows the results (mean±SD; n=4) of selected pharmacokinetic parameters after dosing with insulin alone, or with 0.2 or 0.62 mg Up₄U.

| | | | |
|---|---|---|---|
| Insulin dose (U/kg) | 10 | 10 | 10 |
| Up₄U dose | — | 0.2 | 0.62 |
| (mg) | | | |
| Up₄U concentration | — | 2.0 mg/mL | 6.2 mg/mL |
| (mg/mL) | | | |
| t_{1/2} (min) | 35.4 ± 3.41 | 79.6 ± 42.61 | 47.9 ± 2.23 |
| Cₘₐₓ (μU/mL) | 527 ± 42.2 | 531 ± 89.5 | 655 ± 46.17 |
| F (%) | 3.75 ± 0.84 | 8.07 ± 3.40 | 5.47 ± 1.03 |

The results from this study demonstrate the ability of P2Y agonists, such as UTP and Up₄U, to increase the half life (t_{1/2}), maximal plasma concentration (Cₘₐₓ) and the overall bioavailable fraction (F) of insulin administered to the lungs.

What is claimed is:

1. A method of increasing the systemic absorption of molecules across the surface of the lung of a subject, said method comprising:
administering to said subject a nucleotide receptor agonist in an amount effective to increase the absorption of molecules across the surface of the lung to the systemic circulation.

2. A method of facilitating the systemic delivery of therapeutic agents across the surface of the lung of a subject, said method comprising:
administering to said subject a nucleotide receptor agonist in an amount effective to facilitate the delivery of therapeutic agents across the surface of the lung.

3. The method according to claim 1 or 2, wherein said nucleotide receptor agonist is a P2Y receptor agonist.

4. The method according to claim 3, wherein said P2Y receptor agonist is a nucleoside diphosphate of Formulae Ia, Ib or Ic: 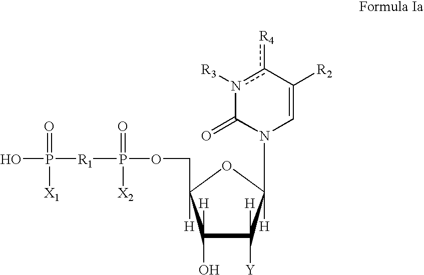
wherein:
X₁, and X₂ are each independently either O— or S—;
Y is H or OH;
R₁ is O, imido, methylene, or dihalomethylene;
R₂ is H, Br, halogen, alkyl, substituted alkyl, alkoxyl, nitro, or azido;
R₃ is nothing, H, alkyl, acyl, arylacyl, or arylalkyl; and
R₄ is —OR′, —SR′, —NR′ or —NR′R″, wherein R′ and R″ are independently H, alkyl, substituted alkyl, aryl, substituted aryl, arylalkyl, alkoxyl, or aryloxyl; provided that when R₄ is double bonded from an oxygen or sulfur atom to the carbon at the 4-position of the pyrimidine ring, R′ is absent; 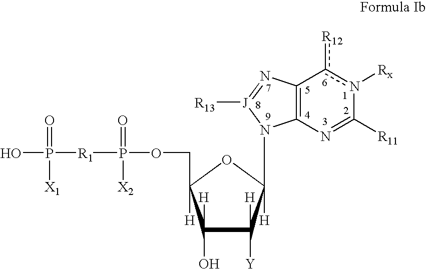
wherein:
R₁, X₁, X₂, and Y are defined as in Formula Ia;
wherein:
R₁₁ is hydrogen, chlorine, amino, monosubstituted amino, disubstituted amino, alkylthio, arylthio, or aralkylthio, where the substituent on sulfur contains up to a maximum of 20 carbon atoms, with or without unsaturation;
R₁₂ is hydroxy, alkenyl, oxo, amino, mercapto, thione, alkylthio, arylthio, aralkylthio, acylthio, alkyloxy, aryloxy, aralkyloxy, acyloxy, monosubstituted alkylamino, heterocyclic, monosubstituted cycloalkylamino, monosubstituted aralkylamino, monosubstituted arylamino, diaralkylamino, diarylamino, dialkylamino, acylamino, or diacylamino;
R_{X} is O, H or absent;
R₁₂ and R_{X} optionally taken together form a 5-membered fused imidazole ring of 1,N⁶-ethenoadenine derivatives, optionally substituted on the 4- or 5-positions of the etheno moiety with alkyl, aryl, nitroaryl, haloaryl, aralkyl, or alkoxy moieties;
R₁₃ is hydrogen, azido, alkoxy, aryloxy, aralkyloxy, alkylthio, arylthio, or aralkylthio, or T(C₁₋₆ alkyl)OCONH(C₁₋₆ alkyl)W— wherein T and W are independently amino, mercapto, hydroxy or carboxyl; or pharmaceutically acceptable esters, amides or salts thereof; or absent;
J is carbon or nitrogen, with the provision that when J is nitrogen, R₁₃ is not present; and
wherein alkyls are straight-chain, branched or cyclic;
wherein aryl groups are substituted with lower alkyl, aryl, amino, mono- or dialkylamino, NO₂, N₃, cyano, carboxylic, amido, sulfonamido, sulphonic acid, phosphate, halo groups, or nothing 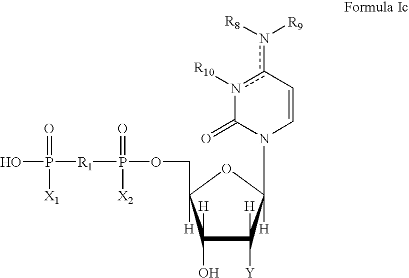
wherein:
R₁, X₁, X₂, and Y are defined as in Formula Ia;
R₈ and R₉ are H while R₁₀ is nothing and there is a double bond between N-3 and C-4, or
R₈, R₉ and R₁₀ taken together are —CH═CH— forming a ring from N-3 to N-4 with a double bond between N-4 and C-4; optionally, the hydrogens of the 4- or 5-position of the etheno ring are independently substituted with alkyl, substituted alkyl, aryl, substituted aryl, alkoxyl, nitro, halo, or azido.

5. The method according to claim 4, wherein said nucleoside diphosphate is 5′-uridine diphosphate, 5′-adenosine diphosphate or 5′-cytidine diphosphate.

6. The method according to claim 3, wherein said P2Y receptor agonist is a nucleoside triphosphate of Formulae IIa, IIb, and IIc: 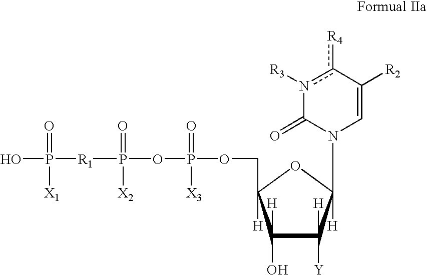
wherein:
X₁, X₂ and X₃ are each independently either O⁻ or S⁻,
Y is H or OH;
R₁ is O, imido, methylene, or dihalomethylene;
R₂ is H, Br, halogen, alkyl, substituted alkyl, alkoxyl, nitro, or azido;
R₃ is nothing, H, alkyl, arylalkyl, acyl, arylacyl, or arylalkyl; and
R₄ is —OR′, —SR′, NR′, or NR′R″, wherein R′ and R″ are independently H, alkyl, substituted alkyl, aryl, substituted aryl, arylalkyl, alkoxyl, or aryloxyl; provided that when R₄ is double bonded from an oxygen or sulfur atom to the carbon at the 4-position of the pyrimidine ring, R′ is absent; 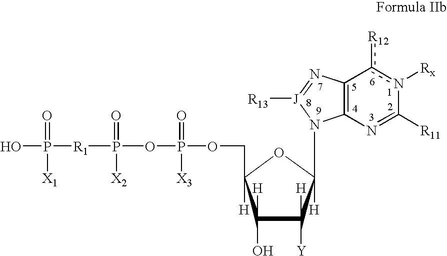
wherein:
R₁, X₁, X₂, X₃, and Y are defined as in Formula IIa;
wherein:
R₁₁ is hydrogen, chlorine, amino, monosubstituted amino, disubstituted amino, alkylthio, arylthio, or aralkylthio, where the substituent on sulfur contains up to a maximum of 20 carbon atoms, with or without unsaturation;
R₁₂ is hydroxy, alkenyl, oxo, amino, mercapto, thione, alkylthio, arylthio, aralkylthio, acylthio, alkyloxy, aryloxy, aralkyloxy, acyloxy, monosubstituted alkylamino, heterocyclic, monosubstituted cycloalkylamino, monosubstituted aralkylamino, monosubstituted arylamino, diaralkylamino, diarylamino, dialkylamino, acylamino, or diacylamino;
R_{X} is O, H or absent;
R₁₂ and R_{X} optionally taken together form a 5-membered fused imidazole ring of 1,N⁶-ethenoadenine derivatives, optionally substituted on the 4- or 5-positions of the etheno moiety with alkyl, aryl, nitroaryl, haloaryl, aralkyl, or alkoxy moieties;
R₁₃ is hydrogen, azido, alkoxy, aryloxy, aralkyloxy, alkylthio, arylthio, or aralkylthio, or T(C₁₋₆ alkyl)OCONH(C₁₋₆ alkyl)W— wherein T and W are independently amino, mercapto, hydroxy or carboxyl; or pharmaceutically acceptable esters, amides or salts thereof; or absent;
J is carbon or nitrogen, with the provision that when J is nitrogen, R₁₃ is not present; and
wherein alkyls are straight-chain, branched or cyclic;
wherein aryl groups are substituted with lower alkyl, aryl, amino, mono- or dialkylamino, NO₂, N₃, cyano, carboxylic, amido, sulfonamido, sulphonic acid, phosphate, halo groups, or nothing; 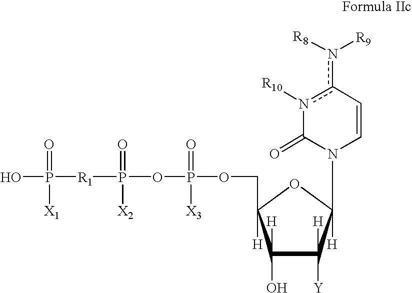
wherein:
R₁, X₁, X₂, X₃, and Y are defined as in Formula Ia;
R₈ and R₉ are H while R₁₀ is nothing and there is a double bond between N-3 and C-4; or
R₈, R₉ and R₁₀ taken together are —CH═CH—, forming a ring from N-3 to N-4 with a double bond between N-4 and C-4; optionally, the hydrogens of the 4- or 5-position of the etheno ring are independently substituted with alkyl, substituted alkyl, aryl, substituted aryl, alkoxyl, nitro, halo, or azido.

7. The method according to claim 6, wherein said nucleoside triphosphate is uridine 5′-triphosphate, adenosine 5′-triphosphate, cytidine 5′-triphosphate, or 4-nitrophenylethenocytidine 5′-triphosphate.

8. The method according to claim 3, wherein said P2Y receptor agonist is a dinucleoside polyphosphate of Formula III: 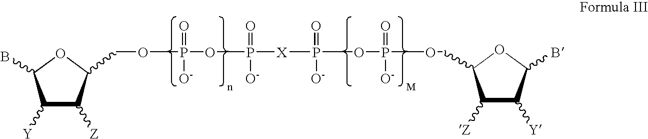
wherein:
X is oxygen, methylene, dihalomethylene, or imido;
n=0, 1 or 2;
m=0, 1 or 2;
n+m=0, 1, 2, 3 or 4;
Z=H or OH;
Z′=H or OH;
Y=H or OH;
Y′=H or OH; and
B and B′ are each independently a purine residue or a pyrimidine residue, as defined in Formula IIIa and IIIb, respectively, linked through the 9- or 1-position, respectively: 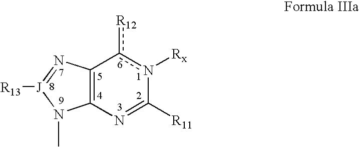
wherein:
R₁₁ is hydrogen, chlorine, amino, monosubstituted amino, disubstituted amino, alkylthio, arylthio, or aralkylthio, where the substituent on sulfur contains up to a maximum of 20 carbon atoms, with or without unsaturation;
R₁₂ is hydroxy, alkenyl, oxo, amino, mercapto, thione, alkylthio, arylthio, aralkylthio, acylthio, alkyloxy, aryloxy, aralkyloxy, acyloxy, monosubstituted alkylamino, heterocyclic, monosubstituted cycloalkylamino, monosubstituted aralkylamino, monosubstituted arylamino, diaralkylamino, diarylamino, dialkylamino, acylamino, or diacylamino;
R_{X} is O, H or absent;
R₁₂ and R_{X} optionally taken together form a 5-membered fused imidazole ring of 1,N⁶-ethenoadenine derivatives, optionally substituted on the 4- or 5-positions of the etheno moiety with alkyl, aryl, nitroaryl, haloaryl, aralkyl or alkoxy moieties;
R₁₃ is hydrogen, azido, alkoxy, aryloxy, aralkyloxy, alkylthio, arylthio, or aralkylthio, or T(C₁₋₆ alkyl)OCONH(C₁₋₆ alkyl)W— wherein T and W are independently amino, mercapto, hydroxy or carboxyl; or pharmaceutically acceptable esters, amides or salts thereof; or absent;
J is carbon or nitrogen, with the provision that when J is nitrogen, R₁₃ is not present; and
wherein alkyls are straight-chain, branched or cyclic;
wherein aryl groups are substituted with lower alkyl, aryl, amino, mono- or dialkylamino, NO₂, N₃, cyano, carboxylic, amido, sulfonamido, sulphonic acid, phosphate, halo groups, or nothing; 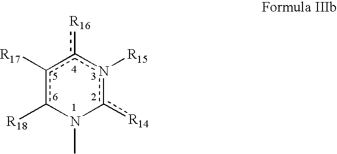
wherein:
R₁₄ is oxo, hydroxy, mercapto, thione, amino, cyano, C₇₋₁₂ arylalkoxy, C₁₋₆ alkylthio, C₁₋₆ alkoxy, C₁₋₆ alkylamino or diC₁₋₄ alkylamino, wherein the alkyl groups are optionally linked to form a heterocycle;
R₁₅ is hydrogen, acetyl, benzoyl, C₁₋₆ alkyl, C₁₋₅ alkanoyl, aroyl, or absent;
R₁₆ is hydroxy, oxo, mercapto, thione, C₁₋₄ alkoxy, C₇₋₁₂ arylalkoxy, C₁₋₆ alkylthio, S-phenyl, arylthio, aralkylthio, arylalkylthio, triazolyl, amino, C₁₋₅ disubstituted amino, C₁₋₆ alkylamino, or di-C₁₋₄ alkylamino wherein said dialkyl groups are optionally linked to form a heterocycle or linked to form a substituted ring; or
R₁₅ and R₁₆ taken together form a 5-membered fused imidazole ring of 3,N⁴-ethenocytosine derivatives between positions 3 and 4 of the pyrimidine ring, wherein said etheno moiety is optionally substituted on the 4- or 5-positions with C₁₋₄ alkyl, phenyl, phenyloxy, or nitrophenyl; wherein at least one hydrogen of said C₁₋₄ alkyl, phenyl or phenyloxy is optionally substituted with halogen, hydroxy, C₁₋₄ alkoxy, C₁₋₄ alkyl, C₆₋₁₀ aryl, C₇₋₁₂ arylalkyl, carboxy, cyano, nitro, sulfonamido, sulfonate, phosphate, sulfonic acid, amino, C₁₋₄ alkylamino, or di-C₁₋₄ alkylamino wherein said dialkyl groups are optionally linked to form a heterocycle;
R₁₇ is hydrogen, hydroxy, cyano, nitro, C₁₋₆ alkyl, phenyl, substituted C₂₋₈ alkynyl, halogen, substituted C₁₋₄alkyl, CF₃, C₂₋₃ alkynyl, allylamino, bromovinyl, ethyl propenoate, propenoic acid, or C₂₋₈ alkenyl; or
R₁₆ and R₁₇ together form a 5 or 6-membered saturated or unsaturated ring bonded through N or O or S at R₁₆, said ring optionally contains functional substituents; and
R₁₈ is hydrogen, amino, di-C₁₋₄ alkylamino, C₁₋₄ alkoxy, C₇₋₁₂ arylalkoxy, C₁₋₄ alkylthio, C₇₋₁₂ arylalkylthio, carboxamidomethyl, carboxymethyl, methoxy, methylthio, phenoxy, or phenylthio; provided that when R₁₈ is amino or substituted amino, R₇ is hydrogen.

9. The method according to claim 4, 6 or 8, wherein the sugar moiety is a ribosyl or deoxyribosyl moiety.

10. The method according to claim 9, wherein the sugar moiety is selected from the group consisting of: ribofuranosyl, 2′-deoxyribofuranosyl, 3′-deoxyfuranosyl, 2′,3′-dideoxyribofuranosyl, arabinofuranosyl, 3′-deoxyarabinofuranosyl, xylofuranosyl, 2′-deoxyxylofuranosyl and lyxofuranosyl.

11. The method according to claim 8, wherein said dinucleoside polyphosphates of general Formula III are dinucleoside tetraphosphates selected from the group consisting of P¹P⁴-di(uridine 5′-)tetraphosphate; P¹-(cytosine 5′)-P⁴-(uridine 5′)tetraphosphate; P¹,P⁴-di(adenosine 5′-)tetraphosphate; P¹-(adenosine 5′)-P⁴-(uridine 5′-)tetraphosphate; P¹-(adenosine 5′)-P⁴-(cytosine 5′-)tetraphosphate; P¹,P⁴-di(ethenoadenosine)tetraphosphate; P¹-(uridine 5′-)-P⁴-(thymidine 5′-) tetraphosphate; P¹-(adenosine 5′)-P⁴-(inosine 5′-)tetraphosphate; P¹,P⁴-di(uridine 5′-)P²,P³-methylenetetraphosphate; P¹,P⁴-di(uridine 5′-P²,P³-difluoromethylenetetraphosphate); P¹,P⁴-di(uridine 5-P²,P³-imidotetraphosphate); P¹,P⁴-di(4-thiouridine 5′-tetraphosphate); P¹,P⁴-di(3,N⁴-ethenocytidine 5′-) tetraphosphate; P¹,P⁴-di(imidazo[1,2-c]pyrimidine-5(6H)-one-2-(3-nitro)-phenyl-6-O-D-ribofuranoside 5′-)tetraphosphate, tetraammonium salt; P¹-(inosine 5′-)P⁴-(uridine 5′-)tetraphosphate; P¹-(4-thiouridine 5′-)P⁴-(uridine 5′-)tetraphosphate; P¹-(cytosine β-D-arabinofuranoside 5′-)P⁴-(uridine 5′-) tetraphosphate; P¹-(uridine 5′-) P⁴-(xanthosine 5′-)tetraphosphate; P¹-(2′-deoxyuridine 5′-)-P⁴-(uridine 5′-) tetraphosphate; P¹-(3′-azido-3′-deoxythyrmidine 5′-)-P⁴-(uridine 5′-)tetraphosphate; P¹,P⁴-di(3′-azido-3′-deoxythymidine 5′-)tetraphosphate₂P₄; P¹,P⁴-di(3′-azido-3′-deoxythymidine 5′-)tetraphosphate; 2′(3′)-benzoyl-P¹,P⁴-di(uridine 5′-)tetraphosphate; P¹,P⁴-di(2′(3′)-benzoyl uridine 5′-) tetraphosphate; P¹-(2′-deoxyguanosine 5′-)P⁴-(uridine 5′-)tetraphosphate; P¹-(2′-deoxyadenosine 5′-)P⁴-(uridine 5′-)tetraphosphate; P¹-(2′-deoxyinosine 5′-)P⁴-(uridine 5′-)tetraphosphate; P¹-(2′-deoxycytidine 5′)P⁴-(uridine 5′-)tetraphosphate; P¹-(4-thiouridine 5′-)P⁴-(uridine 5′-)tetraphosphate; P¹-(8-azaadenosine-5′-)P⁴-(uridine 5′-) tetraphosphate; P¹-(6-mercaptopurine riboside 5′-)P⁴-(uridine 5′-)tetraphosphate; P¹-(6-mercaptopurine riboside 5′-)P⁴-(2′-deoxyuridine 5′-)tetraphosphate; P¹-(4-thiouridine 5′-)P⁴-(arabinocytidine 5′-)tetraphosphate; P¹-(adenosine 5′-)P⁴-(4-thiomethyluridine 5′-) tetraphosphate; P¹-(2′-deoxyadenosine 5′-)P⁴-(6-thiohexylpurine riboside 5′-) tetraphosphate, and P¹-(6-eicosanyloxypurine riboside 5′-)P⁴-(uridine 5′-)tetraphosphate.

12. The method according to claim 8, wherein said dinucleoside polyphosphates of general Formula III are dinucleoside triphosphates selected from a group consisting of: P¹P³-di(uridine 5′-)triphosphate; P¹-(cytosine 5′)-P³-(uridine 5′-)triphosphate; P¹,P³-di(adenosine 5′-)triphosphate; P¹-(adenosine 5′)-P³-(uridine 5′-)triphosphate; P¹-(adenosine 5′)-P³-(cytosine 5′-)triphosphate; P¹,P³-di(ethenoadenosine)triphosphate; P¹-(uridine 5′)-P³-(thymidine 5′-)triphosphate; P¹-(adenosine 5′)-P³-(inosine 5′-)triphosphate; P¹,P³-di(uridine 5′-)P²,P³-methylenetriphosphate; P¹,P³-di(uridine 5′-P²,P³-difluoromethylenetriphosphate); P¹,P³-di(uridine 5′-P²,P³-iridotriphosphate); P¹,P³-di(4-thiouridine 5′-triphosphate); P¹,P³-di(3,N⁴-ethenocytidine 5′-)triphosphate; P¹,P³-di(imidazo[1,2-c]pyrimidine-5(6H)-one-2-(3-nitro)-phenyl-6-β-D-ribofuranoside 5′-)triphosphate, tetraammonium salt; P¹-(inosine 5′-)P³-(uridine 5′-)triphosphate; P¹-(4-thiouridine 5′-)P³-(uridine 5′-) triphosphate; P¹-(cytosine β-D-arabinofuranoside 5′-)P³-(uridine 5′) triphosphate; P¹-(uridine 5′-)P³-(xanthosine 5′-)triphosphate; P¹-(2′-deoxyuridine 5′-)-P³-(uridine 5′-)triphosphate; P¹-(3′-azido-3′-deoxythymidine 5′-)-P³-(uridine 5′-) triphosphate; P¹,P³-di(3′-azido-3′-deoxythymidine 5′-)triphosphate; P¹,P³-di(3′-azido-3′-deoxythymidine 5′-)triphosphate; 2′(3′)-benzoyl-P¹,P³-di(uridine 5′-)triphosphate; P¹,P³-Di(2′(3′)-benzoyl uridine 5′-) triphosphate; P¹-(2′-deoxyguanosine 5′-)P³-(uridine 5′-)triphosphate; P¹-(2′-deoxyadenosine 5′-)P³-(uridine 5′-)triphosphate; P¹-(2′-deoxyinosine 5′-)P³-(uridine 5′-)triphosphate; P¹-(2′-deoxycytidine 5′-)P³-(uridine 5′-)triphosphate; P¹-(4-thiouridine 5′-)P³-(uridine 5′-)triphosphate; P¹-(8-azaadenosine-5′-)P³-(uridine 5′-) triphosphate; P¹-(6-mercaptopurine riboside 5′-)P³-(uridine 5′-)triphosphate; P¹-(6-mercaptopurine riboside 5′-)P³-(2′-deoxyuridine 5′-)triphosphate; P¹-(4-thiouridine 5′-)P³-(arabinocytidine 5′-)triphosphate; P¹-(adenosine 5′-)P³-(4-thiomethyluridine 5′-) triphosphate; P¹-(2′-deoxyadenosine 5′-)P³-(6-thiohexylpurine riboside 5′-) tetraphosphate; and P¹-(6-eicosanyloxypurine riboside 5′-)P³-(uridine 5′-) triphosphate.

13. The method according to claim 8, wherein said dinucleoside polyphosphates of general Formula III are selected from a group consisting of: P¹-(uridine 5′-)P²-(4-thiouridine 5′-) diphosphate; P¹,P⁵-di(uridine 5′-)pentaphosphate; and P¹,P⁶-di(uridine 5′-)hexaphosphate.

14. The method according to claim 1 or 2, wherein said nucleotide receptor agonist is administered in a sterile pharmaceutical composition comprising said nucleotide receptor agonist or pharmaceutically acceptable salts thereof, together with a pharmaceutically suitable carrier.

15. The method according to claim 1 or 2, wherein said nucleotide receptor agonist is co-administered with a therapeutic agent.

16. The method according to claim 15, wherein said therapeutic agent is selected from the group consisting of a protein, hormone, nucleic acid, virus, antimicrobial agent, antiviral agent, analgesic agent, anti-inflammatory agent, anti-neovascular agent, neuroprotectant, anti-depressant, and respiratory agent.

17. The method according to claim 16, wherein said protein is selected from the group consisting of insulin, alpha interferon, beta interferon, human growth hormone, granulocyte cell stimulating factor, epoetin alpha, epoetin beta, entanercept, aglucerase, filgrastim, lenograstim, pegaspargase, sargramostim, interleukin, calcitonin, heparin, follicle stimulating hormone, progesterone, luprolide, estrogen and somatrem.

18. The method according to claim 1 or 2, wherein said nucleotide receptor agonist is co-administered with a diagnostic agent.

19. The method according to claim 18, wherein said diagnostic agent is selected from the group consisting of contrast agents, diagnostic imaging agents and radiolabeled compounds.

20. The method according to claim 14, wherein said administering is systemic administration of a form selected from the group consisting of: an aerosol suspension of respirable particles; a liquid or liquid suspension for administration as nose drops or nasal spray; a nebulized liquid for administration to oral or nasopharyngeal airways; an oral form; an injectable form; a suppository form; and a transdermal patch or a transdermal pad; such that a therapeutically effective amount of said compound contacts the airway epithelium of said subject via systemic absorption and circulation.

21. The method according to claim 14, wherein said administering is direct intra-operative instillation of a form selected from the group comprising a gel, cream, and liquid suspension form of a therapeutically effective amount of the active compound.

22. The method according to claim 14, wherein said administering is by bronchiolar lavage.

23. A nucleotide receptor agonist for use in a method of increasing the systemic absorption of molecules across the surface of the lung of a subject comprising administering to said subject the nucleotide receptor agonist in an amount effective to increase the absorption of molecules across the surface of the lung to the systemic circulation.

24. A nucleotide receptor agonist for use in a method of facilitating the systemic delivery of therapeutic agents across the surface of the lung of a subject comprising administering to said subject a nucleotide receptor agonist in an amount effective to facilitate the delivery of therapeutic agents across the surface of the lung.

25. A nucleotide receptor agonist according to claim 23 or claim 24 as defined in any one of claims 3 to 22.

26. A nucleotide receptor agonist according to any one of claims 23 to 25 in a sterile pharmaceutical composition together with a pharmaceutically suitable carrier.

27. A nucleotide receptor agonist according to any one of claims 23 to 25 in combination with a therapeutic agent.

28. A nucleotide receptor agonist according to claim 27 wherein the therapeutic agent is as defined in claim 16 or claim 17.

29. A nucleotide receptor agonist according to any one of claims 23 to 25 in combination with a diagnostic agent.

30. A nucleotide receptor agonist according to claim 29 wherein the diagnostic agent is as defined in claim 19.

31. A nucleotide receptor agonist according to claim 26 for use in a method according to any one of claims 20 to 22.

32. Use of a compound according to claim 23 or to any one of claims 25 to 31 as dependent on claim 23 in the manufacture of a medicament for increasing the systemic absorption of molecules across the surface of the lung of a subject.

33. Use of a compound according to claim 24 or to any one of claims 25 to 31 as dependent on claim 24 in the manufacture of a medicament for facilitating the systemic delivery of therapeutic agents across the surface of the lung of a subject.
